(19) United States
(12) Patent Application Publication
(10) Pub. No.: US 2004/0235813 A1
(43) Pub. Date: Nov. 25, 2004

(54) Compounds that inhibit hsp90 and stimulate hsp70 and hsp40, useful in the prevention or treatment of diseases associated with protein aggregation and amyloid formation

(76) Inventors: Erich Wanker, Berlin (DE); Annie Sittler, Strasbourg (DE); Ulrich Hartl, Kottgeisering (DE)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/476,812

(22) PCT Filed: May 3, 2002
(86) PCT No.: PCT/EP02/04893

Related U.S. Application Data
(60) Provisional application No. 60/288,718, filed on May 4, 2001.

(30) Foreign Application Priority Data
May 3, 2001 (EP) 01110769.5

(51) Int. Cl. A61K 31/33

(52) U.S. Cl. 514/183

(57) ABSTRACT

The present invention relates to the use of a compound or a plurality of compounds that inhibit function of Hsp90; or activate expression of both Hsp40 and Hsp70 for the preparation of a pharmaceutical composition for the prevention or treatment of a disease associated with protein aggregation and amyloid formation. Preferably, said compound is geldanamycin. The present invention relates further to methods of producing compounds within proved potency and/or decreased side-effects that may be successfully employed as medicaments for the treatment of said diseases.

The present invention relates to the use of a compound or a plurality of compounds that inhibit function of Hsp90; or activate expression of both Hsp40 and Hsp70 for the preparation of a pharmaceutical composition for the prevention or treatment of a disease associated with protein aggregation and amyloid formation. Preferably, said compound is geldanamycin. The present invention relates further to methods of producing compounds within proved potency and/or decreased side-effects that may be successfully employed as medicaments for the treatment of said diseases.

Although since the cloning of the Huntington's disease (HD) gene significant advances have been made in the understanding of the molecular mechanisms underlying this neurodegenerative disease, there is still no effective treatment for HD. HD is caused by an unstable CAG trinucleotide repeat expansion located in the exon 1 of the IT-15 gene encoding huntingtin, a ˜350 kDa protein of unknown function (1-3). Evidence has been presented that the formation of neuronal inclusions with aggregated huntingtin protein is associated with the progressive neuropathology in HD (4). However, it is unclear today whether the process of aggregate formation is the cause of HD or merely a consequence of this disorder (5-7). Using in vitro model systems it was demonstrated recently, that the formation of huntingtin protein aggregates critically depends on polyglutamine repeat length, protein concentration and time (8,9). Furthermore, formation of insoluble aggregates with a fibrillar amyloid-like morphology can be inhibited by small chemical compounds such as Congo red and thioflavine S and the monoclonal antibody 1C2 that specifically recognizes an elongated polyglutamine tract (10). This suggested that inhibition of huntingtin protein aggregation in patients by small molecules could be a promising therapeutic strategy. Histochemical studies revealed that inclusions containing insoluble polyglutamine-containing protein aggregates in brains of patients and transgenic animals are immunoreactive for ubiquitin, various molecular chaperones and components of the 20 S proteasome (2,11). This suggests that neuronal cells recognize the aggregated huntingtin protein as abnormally folded and by recruiting chaperones and proteasomal components try to disaggregate and/or degrade the mutant protein. Consistent with this view, overexpression of the heat shock proteins Hsp40, Hsp70 and Hsp104 in cell culture, yeast, C. elegans and fly model systems has blocked the accumulation of polyglutamine-containing protein aggregates (12-15). However, whether the formation of insoluble protein aggregates can be suppressed by activation of a heat shock response is unknown.

However, whereas several papers (14, 15, 26) report on a critical involvement Hsp40 and Hsp70 chaperones in the suppression of polyglutamine induced neurodegeneration, these data leave many important questions open and do not allow without further ado for the direct development of medicaments useful in the treatment or prevention of diseases associated with protein aggregation or amyloid formation. For example, Chan and colleagues (14) demonstrated that suppression of neurodegeneration in a Drosophila model may depend on the Hsp40 chaperone involved. In addition, lethality of the flies as a possible result of neurodegeneration was mitigated by chaperone overexpression in a sex-dependent manner. Accordingly, it appears questionable whether the results obtained in the Drosophila systems may easily adapted to a human system. According to Jana and colleagues (15), the challenge for future investigations is to determine whether Hsp40 and Hsp70 family chaperones really suppress the aggregation and protect neurodegeneration in poly Q related diseases such as Huntington disease. Should this indeed turn out to be the case, then Jana et al. suggest to directly use such chaperones as therapeutic agents for the treatment of said diseases. Furthermore, the previous findings that Hsp40 and Hsp70 are able to suppress polyglutamine aggregation in a Drosophila and cell culture model can not easily be used for the therapy of neurodegenerative diseases, because gene therapy in human patients has been shown to be very problematic. Therefore, in order to use the expression of heat shock proteins for therapy it is necessary to find small molecules that are nontoxic and penetrate the blood-brain barrier, and that efficiently activate a heat shock response in patients. Such molecules have not been described yet.

Accordingly, there remains a need in the art to provide a suitable approach for the effective prevention/treatment of diseases associated with protein aggregation and amyloid formation.

The solution to this technical problem is achieved by providing the embodiments characterized in the claims.

Accordingly, the present invention relates to the use of a compound or a plurality of compounds that inhibit function of Hsp90; or inhibit binding of HSF1 to Hsp90; or activate expression of both Hsp40 and Hsp70 for the preparation of a pharmaceutical composition for the prevention or treatment of a disease associated with protein aggregation and amyloid formation.

The term “HSF1” refers to the heat shock transcription factor described, e.g. in Zou (25) and references cited therein.

The term “Inhibit function” throughout this specification means an inhibition of at least 30% of the function, preferably at least 50%, more preferred at least 70%, even more preferred at least 90% and most preferred more than 95% such as 98% or even more than 99%.

In accordance with the present invention, it was surprisingly found that compounds, preferably small molecules, that inhibit the function of Hsp90 may effectively be used in the prevention of protein aggregation and amyloid formation and may, thus, successfully be employed in diseases caused by the recited phenomena. This result was not to be expected since the involvement of Hsp90 in the formation of protein aggregation or amyloid formation has so far not been shown in the art. Similarly, it was surprising to find that one compound, preferably a small molecule, is able to simultaneously activate expression of Hsp40 and Hsp70 and consequently form a basis for the prevention or treatment of the referenced diseases. Comprised by the present invention are also uses wherein the compound or compounds both inhibit function of Hsp90 and activate expression of Hsp40 and Hsp70. In accordance with the present invention, the term “function of Hsp90” is intended to mean the function including or consisting of ATPase activity of Hsp90. In accordance with the present invention it is expected that inhibition of ATPase activity results in a dissociation of the ATPase/HFS1 complex whereupon HSF1 migrates into the nucleus and activates expression of Hsp40 and Hsp70. These proteins, in turn, bind to the mutated huntingtin protein and prevent protein aggregation. It is also to be understood that each compound of the plurality of compounds either inhibits function of Hsp90 or simultaneously activates expression of Hsp40 and Hsp70.

Accordingly, the present invention provides an entirely different solution to the approach of developing a medicament useful in the prevention or treatment of diseases associated with protein aggregation or amyloid formation than was suggested by Jana et al., supra. Whereas Jana et al. suggest to directly use chaperones of the Hsp40 and Hsp70 family as therapeutically active agents, the present invention chooses a different approach: namely, the solution underlying the present invention is to provide molecules that modulate function or the expression pattern of the above indicated chaperones. In so far, the approach taken by the present invention is much more amenable to the actual preparation of a medicament since small compounds may be selected that fulfil the above requirements.

Further in accordance with the present invention, either single compounds or a plurality of compounds (with the definition of activity as provided above) may form the active ingredients of the pharmaceutical compositions produced. If more than one compound forms the active ingredient, then the pharmacological effect should be enhanced. For example, it may be additive or synergistic.

Preferred in accordance with the use of the invention is that said disease is associated with polyglutamine expansions.

In a further preferred embodiment of the use of the invention said compound is geldanamycin.

Geldanamycin (GA) is a naturally occuring antitumor drug that has been shown to be active in tumor cell lines as well as in mouse models (16). The antitumor effects of GA result from its ability to deplete cells from proto-oncogenic protein kinases and nuclear hormone receptors (17-19). Initially it was thought that GA is a nonspecific protein kinase inhibitor. However, subsequent biochemical and structural studies have demonstrated that GA binds specifically to the heat shock protein Hsp90, thereby inhibiting its chaperone function (20-22). Hsp90 is specifically involved in folding and conformational regulation of several medically relevant signal transduction molecules, including nuclear receptors and proto-oncogenic kinases (18,23). Inhibition of Hsp90 function by GA causes degradation of these regulatory proteins (18,24). Recently, Zou et al. (25) have shown that GA also disrupts a complex consisting of Hsp90 and the heat shock transcription factor HSF1 and triggers the activation of a heat shock response in mammalian cells. It was particularly surprising in accordance with the present invention that this compound is also useful for the effective treatment of the above recited diseases. Specifically, it could be shown that geldanamycin (GA) exerts a negative effect on the formation of insoluble huntingtin exon 1 aggregates in a cell culture model of HD and thus forms a basis for an active ingredient of a medicament effective in the treatment of diseases associated with protein aggregation and amyloid formation. In particular, it was found that treatment of cells with GA leads to enhanced expression of both Hsp40 and Hsp70 which has a direct implication and appears to be necessary for inhibition of huntingtin protein aggregation which is exemplary of the above recited class of diseases. Although it is state of the art that GA bind to Hsp90 and is able to modulate HSP function, it was absolutely unpredictable whether treatment of cells with GA activates a heat shock response that is sufficient to prevent polyglutamine aggregation.

In another preferred embodiment of the use of the invention said plurality of compounds comprises geldanamycin.

In a further preferred embodiment of the use of the invention said compound or one of said compounds comprised in said plurality of compounds is derived from geldanamycin by modeling geldanamycin by peptidomimetics; and chemically synthesizing the modeled compound.

Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of activators of the expression of the polypeptide of the invention can be used for the design of peptidomimetic activators, e.g., in combination with the (poly)peptide of the invention (Rose, Biochemistry 35 (1996), 12933-12944; Rutenber, Bioorg. Med. Chem. 4 (1996), 1545-1558).

In a different preferred embodiment of the use of the invention said compound or one of said compounds comprised in said plurality of compounds are derived from geldanamycin by modification to achieve modified site of action, spectrum of activity, organ specificity, and/or improved potency, and/or decreased toxicity (improved therapeutic index), and/or decreased side effects, and/or modified onset of therapeutic action, duration of effect, and/or modified pharmakinetic parameters (resorption, distribution, metabolism and excretion), and/or modified physico-chemical parameters (solubility, hygroscopicity, color, taste, odor, stability, state), and/or improved general specificity, organ/tissue specificity, and/or optimized application form and route by esterification of carboxyl groups, or esterification of hydroxyl groups with carbon acids, or esterification of hydroxyl groups to, e.g. phosphates, pyrophosphates or sulfates or hemi succinates, or formation of pharmaceutically acceptable salts, or formation of pharmaceutically acceptable complexes, or synthesis of pharmacologically active polymers, or introduction of hydrophilic moieties, or introduction/exchange of substituents on aromates or side chains, change of substituent pattern, or modification by introduction of isosteric or bioisosteric moieties, or synthesis of homologous compounds, or introduction of branched side chains, or conversion of alkyl substituents to cyclic analogues, or derivatisation of hydroxyl group to ketales, acetales, or N-acetylation to amides, phenylcarbamates, or synthesis of Mannich bases, imines, or transformation of ketones or aldehydes to Schiff's bases, oximes, acetales, ketales, enolesters, oxazolidines, thiozolidines; or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

In an additional preferred embodiment of the use of the invention said plurality of compounds comprises at least one of the following: Radicicol, Herbimycin A, Novobiocin and 17-Allylamino, 17-demethoxygeldanamycin and macbecin.

In another preferred embodiment of the use of the invention said compound is obtained by (a) screening an at least partially randomized peptide library and/or chemical compound library for molecules that (aa) inhibit function of Hsp90; or (ab) inhibit binding of HSF1 to Hsp90; or (ac) activate the expression of both Hsp40 and Hsp70, and optionally repeating step (a) one or more times.

The term “partially randomized peptide library” refers to collections of synthetic peptides ranging in numbers from less than 10 to thousands (37, 38). The premise of such libraries is that they enable the identification of complete novel, biologically active peptides through screening without any prior structural and sequence knowledge. Partially randomized peptide libraries contain synthetic peptides which are randomized at specific amino acid positions in the peptides.

Peptide libraries presented to date fall into three broad categories, the difference being the manner in which the sequences are synthesized and/or screened. The first category represents synthetic approaches, in which peptide mixtures are synthesized, cleaved from their support and assayed as free compounds in solution. The second category includes synthetic combinatorial libraries of peptides that are assayed while attached to either plastic, pins, resins beads, or cotton. The third category includes the molecular biology approaches, in which peptides or proteins are present on the surface of filamentous phage particles or plasmids. All these categories are comprised by the use of the present invention.

In a particularly preferred embodiment of the use of the invention inhibition or activation of said heat shock protein(s) is assayed by Reporter assays, immunofluorescence microscopy, a filter retardation assay or ATPase assays.

In a further particularly preferred embodiment of the use of the invention the following-further steps are conducted for obtaining said compound: modeling said compound by peptidomimetics; and chemically synthesizing the modeled compound.

In another particularly preferred embodiment of the use of the invention said compound is further modified to achieve modified site of action, spectrum of activity, organ specificity, and/or improved potency, and/or decreased toxicity (improved therapeutic index), and/or decreased side effects, and/or modified onset of therapeutic action, duration of effect, and/or modified pharmakinetic parameters (resorption, distribution, metabolism and excretion), and/or modified physico-chemical parameters (solubility, hygroscopicity, color, taste, odor, stability, state), and/or improved general specificity, organ/tissue specificity, and/or optimized application form and route by esterification of carboxyl groups, or esterification of hydroxyl groups with carbon acids, or esterification of hydroxyl groups to, e.g. phosphates, pyrophosphates or sulfates or hemi succinates, or formation of pharmaceutically acceptable salts, or formation of pharmaceutically acceptable complexes, or synthesis of pharmacologically active polymers, or introduction of hydrophilic moieties, or introduction/exchange of substituents on aromates or side chains, change of substituent pattern, or modification by introduction of isosteric or bioisosteric moieties, or synthesis of homologous compounds, or introduction of branched side chains, or conversion of alkyl substituents to cyclic analogues, or derivatisation of hydroxyl group to ketales, acetates, or N-acetylation to amides, phenylcarbamates, or synthesis of Mannich bases, imines, or transformation of ketones or aldehydes to Schiff's bases, oximes, acetates, ketales, enolesters, oxazolidines, thiozolidines or combinations thereof.

The invention also relates to a method of designing a drug for the treatment of a disease associated with protein aggregation and amyloid formation identification of the site(s) of a compound that bind(s) to heat shock proteins 40 and/or 70; or identification of site(s) of a compound that bind(s) to the heat shock protein Hsp90 or to HSF1 and/or homologues thereof or other components participating in the regulation of the stress protein response; molecular modeling of both the binding site(s) in the compound and the heat shock protein(s); and modification of the compound to improve its binding specificity for the heat shock protein(s) or HSF1.

All techniques employed in the various steps of the method of the invention are conventional or can be derived by the person skilled in the art from conventional techniques without further ado. Thus, biological assays based on the herein identified nature of the compounds may be employed to assess the specificity or potency of the drugs wherein the increase of one or more activities of the compounds may be used to monitor said specificity or potency. Steps (1) and (2) and (3) can be carried out according to conventional protocols described, for example, as described herein below.

For example, identification of the binding site of said drug by site-directed mutagenesis and chimerical protein studies can be achieved by modifications in the primary sequence, for example, if the compound is a (poly)peptide, that affect the drug affinity; this usually allows to precisely map the binding pocket for the drug. As regards step (2), the following protocols may be envisaged: Once the effector site for drugs has been mapped, the precise residues interacting with different parts of the drug can be identified by combination of the information obtained from mutagenesis studies (step (1)) and computer simulations of the structure of the binding site provided that the precise three-dimensional structure of the drug is known (if not, it can be predicted by computational simulation). If said drug is itself a peptide, it can be also mutated to determine which residues interact with other residues in the compound of interest.

Finally, in step (3) the drug can be modified to improve its binding affinity or its potency and specificity. If, for instance, there are electrostatic interactions between a particular residue of the compound of interest and some region of the drug molecule, the overall charge in that region can be modified to increase that particular interaction.

Identification of binding sites may be assisted by computer programs. Thus, appropriate computer programs can be used for the identification of interactive sites of a putative inhibitor and the polypeptide by computer assisted searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. Modifications of the drug can be produced, for example, by peptidomimetics and other inhibitors can also be identified by the synthesis of peptidomimetic combinatorial libraries through successive chemical modification and testing the resulting compounds.

Compounds binding with improved specificity to Hsp90 or HSF1 are expected to increase the dissociation of Hsp90 and HSF1.

In a preferred embodiment of the method of the invention identification of binding site(s) in step (a) is effected by site-directed mutagenesis or chimeric protein studies or a combination thereof.

Site-directed mutagenesis and chimeric protein studies are techniques well known in the art and described, forexample, in (39-42).

In another preferred embodiment of the method of the invention the compound is the compound as described in any one of the preceding embodiments.

The invention further relates to a method of identifying an activator of the expression of heat shock proteins 40 and/or 70 comprising testing a compound for the activation of translation wherein said compound is selected from small molecules or peptides; or testing a compound for the activation of transcription wherein said compound binds to the promoter region of the genes encoding said heat shock protein(s) and preferably with transcription factors and responsive elements thereof; and selecting a compound that tests positive in any of the preceding steps.

The term “small molecule” refers to a compound having a relative molecular weight of not more than 1000 D and preferably of not more than 500 D. Said compound may be of differing chemical nature, for example, it may be of proteinaceous nature RNA or DNA.

Additionally, the invention further relates to a method of identifying an inhibitor of Hsp90 function comprising testing a compound for inhibition of Hsp90 ATPase activity function wherein said compound is selected from small molecules or peptides; and selecting a compound that tests positive in the preceding step. In order to select an inhibitor of Hsp90 function mammalian cell lines may be generated which contain reporter constructs with the promoter regions of the genes encoding Hsp90, Hsp40, Hsp70 or HSF1. Then, chemical compounds will be added to cell lines and the activation of a heat shock response will be tested using the reporter constructs. Chemicals which inhibit, for example, Hsp90 ATPase activity should induce the expression of the reporter proteins. The expression of the reporter proteins in cells can, e.g. be monitored by immunofluorescence microscopy, ELISA assays or chemiluminescence. As reporters, proteins such as GFP, β-lactamase or luciferase can be used which are well known in the art. First, derivatives and structural analogues of geldanamycin which are on the basis of the teachings of the invention and the prior art supposed to induce Hsp40 and/or Hsp70 expression will be used to evaluate the reporter assays. Later, the same cell lines will be used to screen libraries of chemical compounds.

In addition, the present invention relates to a method of identifying an inhibitor of binding of HSF1 to Hsp90 comprising testing a compound for inhibition of binding of HSF1 to Hsp90; and selecting a compound that tests positive in the preceding step.

In a preferred embodiment of the method of the invention the method further comprises modeling said compound by peptidomimetics; and chemically synthesizing the modeled compound.

In a further preferred embodiment of the method of the invention said compound is further modified to achieve modified site of action, spectrum of activity, organ specificity, and/or improved potency, and/or decreased toxicity (improved therapeutic index), and/or decreased side effects, and/or modified onset of therapeutic action, duration of effect, and/or modified pharmakinetic parameters (resorption, distribution, metabolism and excretion), and/or modified physico-chemical parameters (solubility, hygroscopicity, color, taste, odor, stability, state), and/or improved general specificity, organ/tissue specificity, and/or optimized application form and route by esterification of carboxyl groups, or esterification of hydroxyl groups with carbon acids, or esterification of hydroxyl groups to, e.g. phosphates, pyrophosphates or sulfates or hemi succinates, or formation of pharmaceutically acceptable salts, or formation of pharmaceutically acceptable complexes, or synthesis of pharmacologically active polymers, or introduction of hydrophilic moieties, or introduction/exchange of substituents on aromates or side chains, change of substituent pattern, or modification by introduction of isosteric or bioisosteric moieties, or synthesis of homologous compounds, or introduction of branched side chains, or conversion of alkyl substituents to cyclic analogues, or derivatisation of hydroxyl group to ketales, acetates, or N-acetylation to amides, phenylcarbamates, or synthesis of Mannich bases, imines, or transformation of ketones or aldehydes to Schiff's bases, oximes, acetates, ketales, enolesters, oxazolidines, thiozolidines or combinations thereof.

In another preferred embodiment of the use of the invention or in a another preferred embodiment of the method of the invention said disease is Creutzfeld Jakob disease, Huntington's disease, spinal and bulbar muscular atrophy, dentarorubral pallidoluysian atrophy, spinocerebellar ataxia type-1, -2, -3, -6 or -7, Alzheimer disease, BSE, primary systemic amyloidosis, secondary systemic amyloidosis, senile systemic amyloidosis, familial amyloid polyneuropathy I, hereditary cerebral amyloid angiopathy, hemodialysis-related amyloidosis, familial amyloid polyneuropathy III, Finnish hereditary systemic amyloidosis, type II diabetes, medullary carcinoma of the thyroid, spongiform encephalopathies: Kuru, Gerstmann-Straussler-Scheinker syndrome (GSS), familial insomnia, scrapie, atrial amyloidosis, hereditary non-neuropathic systemic amyloidosis, injection-localized amyloidosis, hereditary renal amyloidosis, or Parkinson's disease.

In a different embodiment the invention relates to a method of producing a pharmaceutical composition comprising formulating the compound described in the use of the invention or the method of the invention with a pharmaceutically acceptable carrier or diluent.

The invention in yet another embodiment relates to a method or to a use described in the invention wherein said heat shock protein is/said heat shock proteins are human heat shock proteins.

Finally, the invention additionally relates to a method of the invention wherein the human heat shock protein 40 is Hdj-1 or Hdj-2.

The pharmaceutical composition produced in accordance with the present invention may further comprise a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholiclaqueous solutions, emulsions or suspensions, including saline and buffered media. Parenteral vehicles include sodium chloride solution, Ringer's dextrose, dextrose and sodium chloride, lactated Ringers, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

The specification recites a number of documents. The disclosure content of said documents is herewith incorporated by reference.

The figures show:

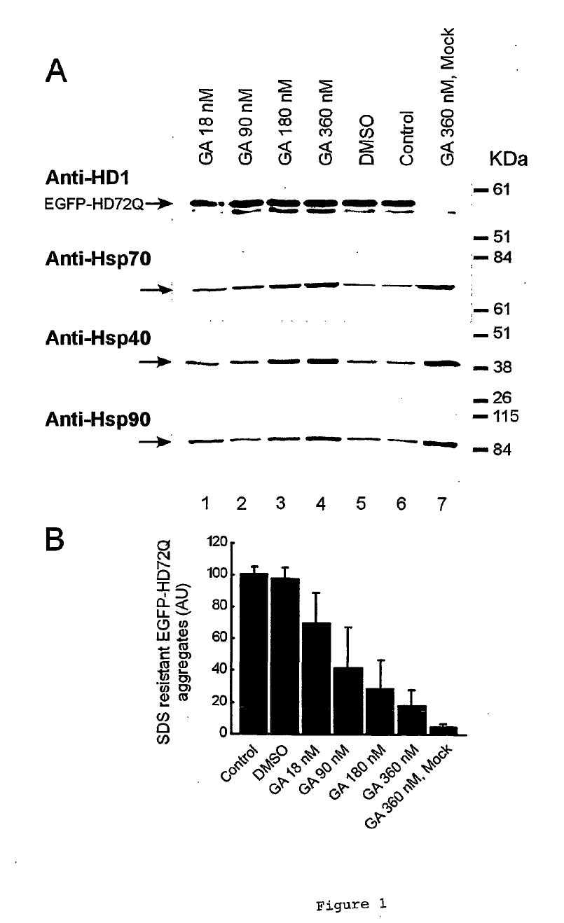

FIG. 1: GA induces a heat shock response and inhibits aggregation of EGFP-HD72Q in COS-1 cells.

(A) Expression of EGFP-HD72Q, Hsp40, Hsp70, and Hsp90 in COS-1 cells. Cells expressing pEGFP-HD72Q were treated for 40 hours with increasing concentrations of GA. Protein extracts prepared from GA treated and untreated cells (control) were analyzed by SDS-PAGE and immunoblotting using the indicated antibodies. Equal amounts (10 μg) of protein were loaded. (B) GA treatment of COS-1 cells prevents the formation of SDS-insoluble EGFP-HD72Q protein aggregates. Aggregates were detected using the filter retardation assay. Filters were probed with the HD1 antibody and signal intensities quantified using a Fuji-imager (LAS 2000). The signal intensity obtained from the sample without added GA was arbitrarily set as 100 (control). Values shown are the mean of three independent experiments (±S.E).

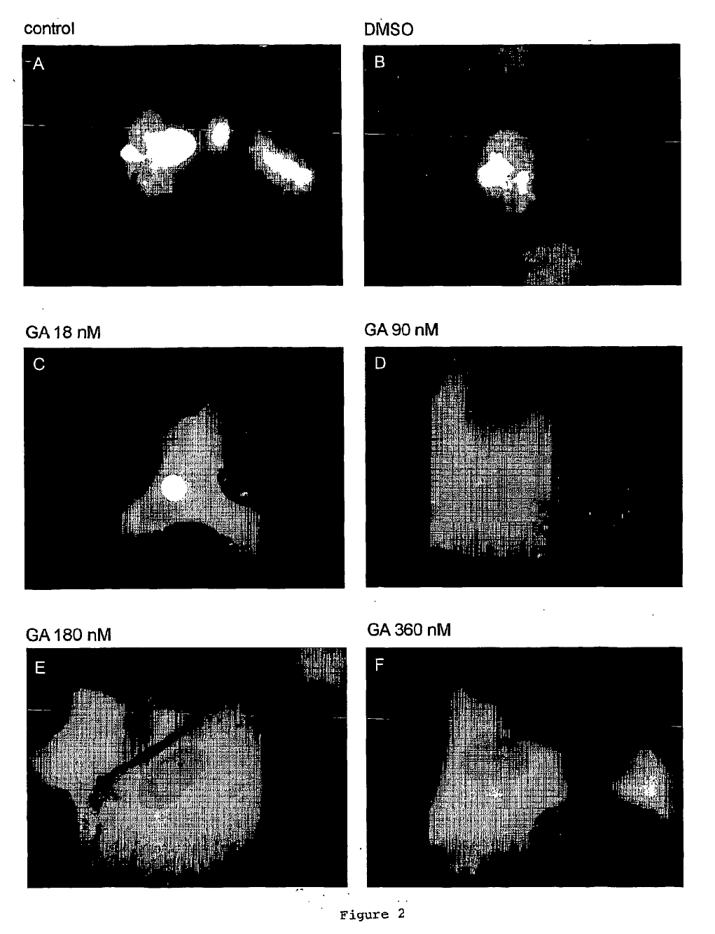

FIG. 2: Fluorescence analysis of GA treated COS-1 cells expressing EGFP-HD 72Q.

COS-1 cells grown for 24 h in the absence (A-B) or presence of GA (C-F) were examined for EGFP-HD72Q expression by fluorescence microscopy (green). Nuclei were counterstained with Hoechst.

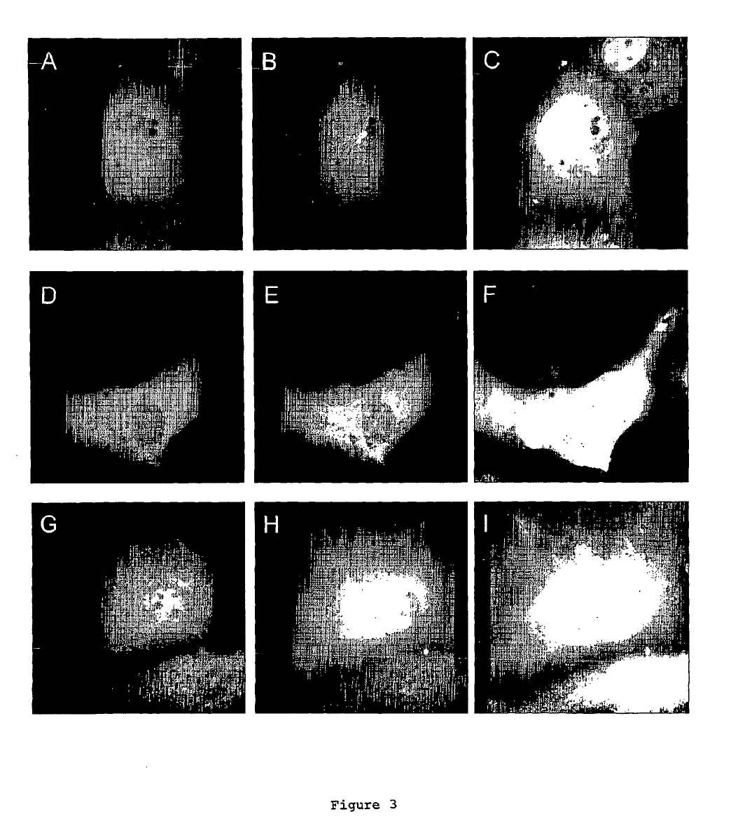

FIG. 3: Co-localization of EGFP-HD72Q with Hsp40, Hsp70 and Hsp90 in GA treated COS-1 cells.

Following incubation of cells for 40 hours with GA at 360 nM, cells expressing EGFP-HD72Q (green) were immunolabeled with antibodies directed against Hsp40 (A-C), Hsp70 (D-F) and Hsp90 (G-l) coupled to a Cy3-conjugated secondary antibody (red). Co-localization of EGFP-HD72Q with Hsp40, Hsp70 and Hsp90 is shown in C, F and I, respectively. Nuclei were counterstained with Hoechst.

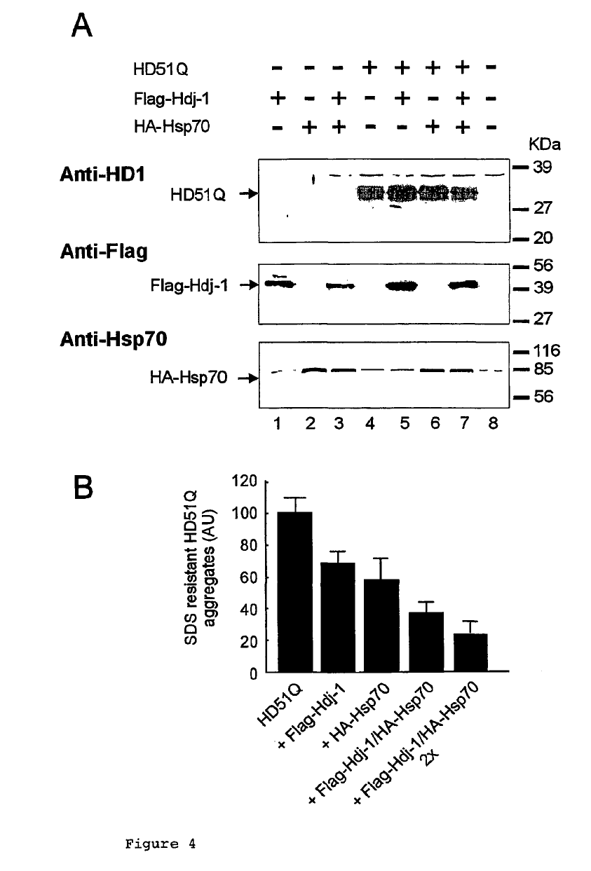

FIG. 4: Overexpression of Flag-Hdj-1 and HA-Hsp70 inhibits HD51Q protein aggregation in COS-1 cells.

(A) Western blot analysis. COS-1 cells were transfected with constructs as indicated on top of the figure. 40 hours post transfection protein extracts were prepared and analyzed by SDS-PAGE and immunoblotting using specific antibodies. Equal amounts (10 μg) of protein were loaded. (B) Inhibition of HD51Q aggregation by overexpression of Flag-Hdj1 and HA-Hsp70. Aggregates were detected and quantified as in FIG. 1B. The signal intensity obtained from the sample without overexpression of heat shock proteins (HD51Q) was arbitrarily set as 100. Data represent means of five independent experiments (±S.E). 2× indicates that the double amount of plasmid DNA was transfected.

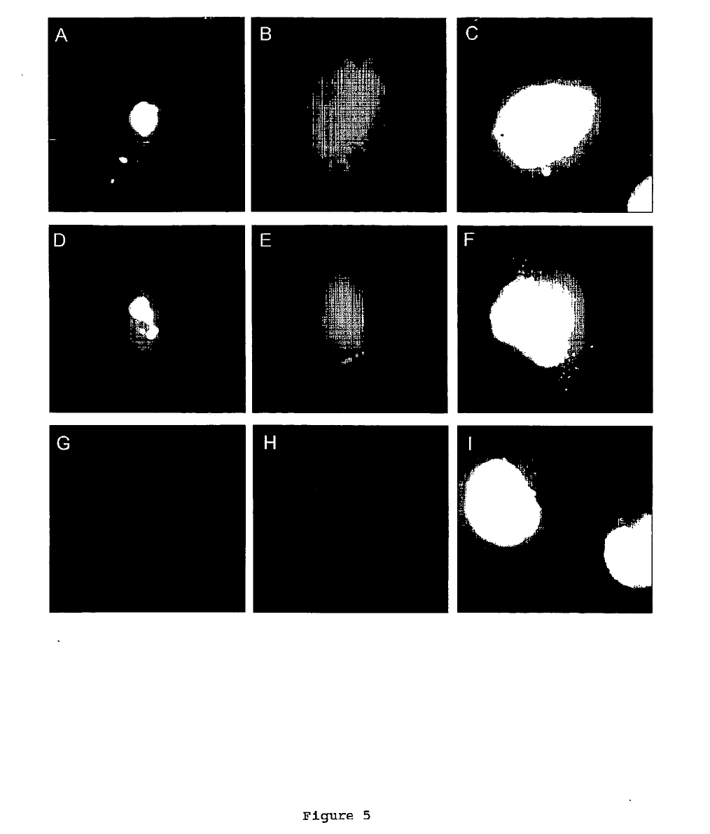

FIG. 5: Immunofluorescence analysis of HD51Q aggregation in COS-1 cells. 42 hours post transfection COS-1 cells co-expressing HD51Q/Flag-Hdj1 (A-C), HD51Q/HA-Hsp70 (D-F) or HD51Q/Flag-Hdj1 /HA-Hsp70 (G-I) were examined by indirect immunofluorescence microscopy. HD51Q protein aggregates were immunolabled with the HD1 antibody coupled to a FITC-conjugated secondary antibody (green). Flag-Hdj1 and HA-Hsp70 were labeled with anti-Flag and anti-Hsp70 antibodies, respectively, coupled to a Cy3-conjugated secondary antibody (red). Nuclei were counterstained with Hoechst.

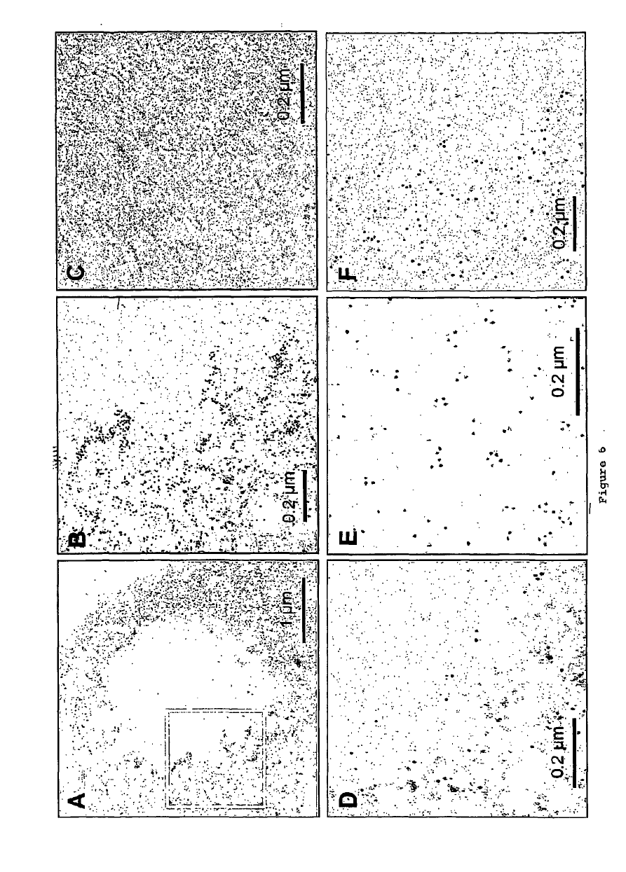

FIG. 6: Ultrastructural analysis of HD51Q aggregates following Flag-Hdj1 and HA-Hsp70 overexpression.

COS-1 cells expressing HD51Q alone (A-C) or co-expressing HD51Q/Flag-Hdj1 (D), HD51Q/HA-Hsp70 (E) or HD51Q/Flag-Hdj1/HA-Hsp70 (F) were viewed by electron microscopy. (A-C) Different magnifications of a cell containing a typical perinuclear inclusion body. At higher magnification HD51Q fibrils can be observed (C). Immunogold labeling of cells with the anti-AG51 antibody confirms the identity of the HD51Q fibrils (B). Immunogold labeling of cells also reveals that Flag-Hdj1 (D) and HA-Hsp70 (E) are associated with HD51Q fibrils. In cells co-expressing HD51Q/Flag-Hdj1/HA-Hsp70 no HD51Q fibrils but homogenous cytoplasmic labeling was observed with the HD1 antibody (F).

The examples illustrate the invention.

### EXAMPLE 1

### Plasmid Constructions

Exon 1 of the human HD gene containing 51 glutamines was derived from pCAG51 (30) and cloned into pTL1 (31) resulting in construct pTL1-CAG51. pTL1-HA was generated by insertion of a Kozak sequence (32) and a sequence encoding a HA-tag (MAYPYDVPDYASLRS) into pTL1. A further linker was introduced in order to generate the appropriate reading frame, resulting in pTL1-HA3. Hsp70-pTLHA3 was generated by PCR amplification of the human Hsp70A gene and cloning into pTL1-HA3. Hdj-1-pTL10Flag was generated by ligating the human HDJ-1 gene, derived from pQE9-His-Hsp40 (33), into pTL10SFlag (a kind gift of D. Devys and J.-L. Mandel). pEGFP-HD72Q was generated by PCR amplification of the exon 1 of human HD from patient DNA and cloning into pEGFP-C1 (Clontech). All constructs were verified by sequencing.

### EXAMPLE 2

### Antibodies

The following antibodies were used for Western blot and/or immunofluorescence analysis: rabbit polyclonal HD1 IgG (30) diluted 1:5000 (WB) or 1:1000 (IF), rabbit polyclonal AG51 IgG (8) diluted 1:100 (immunolabeling in electron microscopy), goat polyclonal anti-Hsp70 (Santa Cruz Biotechnology, Inc.) diluted 1:2000 (WB) or 1:200 (IF), mouse monoclonal anti-Hsp70 (Santa Cruz Biotechnology, Inc.) diluted 1:5000 (WB), rabbit polyclonal anti-Hsp40 (StressGen) diluted 1:10000 (WB) or 1:500 (IF), rabbit polyclonal anti-Hsp90 (Santa Cruz Biotechnology, Inc.) diluted 1:1000 (WB) or 1:100 (IF), mouse monoclonal anti-HA (Boehringer Mannheim) diluted 1:2000 (WB) or 1:200 (IF), and mouse monoclonal M2 anti-Flag (Sigma) diluted 1:10000 (WB) or 1:1000 (IF).

### EXAMPLE 3

### Cell Lines and Cell Transfection

COS-1 cells were grown in Dulbecco's modified Eagle medium (Gibco BRL) supplemented with 5% fetal calf serum (FCS) and containing penicillin (100 U/ml) and streptomycin (100 μg/ml). Transfection was performed by the calcium phosphate co-precipitation technique (34). For the expression of the HD51Q, Flag-Hdj-1 and HA-Hsp70 proteins, cells were plated to 30% confluence in 90 mm plates, and co-transfected with 3 μg of pTL1-CAG51 and 3 or 6 μg of Hsp 70-pTLHA3 and 3 or 6 μg of Hdj-1-pTL10Flag along with 5 or 11 μg of carrier pBluescript DNA. After 16 hours the calcium phosphate precipitate was washed from the cells, and new medium was added on the plates. 40 to 42 hours after transfection the cells were harvested and lysed in presence of protease inhibitors.

Geldanamycin (GibcoBRL Life Technologies, at 1.8 mM stock in DMSO) was diluted into fresh medium to give final concentrations of 18-360 nM and added to cells at the time of transfection. After 16 h cells were washed and new medium containing GA was added. A further medium change with GA was done 24 hours after transfection. Control cells were treated with DMSO. As alternative transfection method, the Lipofectamine Plus Reagent (GibcoBRL Life Technologies) was used according to the manufacturer's instruction.

### EXAMPLE 4

### Preparation of Protein Extracts

Cell lysis and preparation of the soluble and insoluble protein fractions were performed as described (35). For preparation of whole cell extracts cell lysis was performed on ice for 30 min in buffer containing protease inhibitors and nucleic acids were digested with 125 U/mi Benzonase (Merck). Protein concentration was determined by the BioRad assay.

### EXAMPLE 5

### Western Blot Analysis and Filter Retardation Assay

SDS-PAGE and Western blot analysis was performed according to standard procedures. For the filter retardation assay (27,30) protein samples (1-20 μg) were heated at 98° C. for 3 min in 2% SDS and 50 mM DTT and filtered through a 0.2 μm cellulose acetate membrane (Schleicher & Schuell) using a BRL dot-blot filtration unit. Captured aggregates were detected by incubation with HD1 IgG (diluted 1:5000) followed by incubation with alkaline phosphatase conjugated anti-rabbit IgG and the fluorescent substrate AttoPhos. Quantitation of the captured aggregates was performed using a Fuji-imager (LAS 2000) and AIDA 1.0 image analysis software.

### EXAMPLE 6

### Immunofluorescence and Electron Microscopy

Immunofluorescence microscopy of transfected COS-1 was performed as described (35) using the anti-huntingtin HD1 IgG (1:1000) coupled to FITC-conjugated donkey anti rabbit IgG (1:200, Jackson Immuno Research Laboratories), the mouse monoclonal anti-FLAG antibody (1:1000, Sigma) coupled to Cy3-conjugated donkey anti mouse IgG (1:200, Jackson Immuno Research Laboratories), the goat polyclonal anti-Hsp70 antibody (1:200, Santa Cruz Biotechnology, Inc.) coupled to Cy3-conjugated donkey anti goat IgG (1:200, Jackson Immuno Research Laboratories), the anti-Hsp40 (1:500, StressGen) and the anti-Hsp90 (1:300, StressGen) coupled to Cy3-conjugated secondary antibodies. Nuclei were counterstained with Hoechst (bis-benzimide, Sigma). The samples were examined with a fluorescence microscope Axioplan-2 (Zeiss). COS-1 cells transfected with pEGFP-HD72Q were fixed with 2% paraformaldehyde for 4 min at room temperature followed by direct observation of the green fluorescent fusion protein.

For electron microscopic analysis, monolayers of cells were fixed with 1% formaldehyde-0.2% glutaraldehyde for 1 hour, dehydrated in an ethanol series and embedded in LR Gold (London Resin Company, Ldt). Post-embedding immunogold labeling was performed as described (36) using the anti-huntingtin antibodies HD1 (1:400) and AG51 (1:100), or goat anti-Hsp70 (1:400) and goat anti-Hsp40 (1:150) antibodies, followed by secondary antibodies conjugated with 10 nm gold (1:100, British Bio Cell). Sections were poststained with uranyl acetate and lead Citrate. Samples were viewed in a Philips CM100 electron microscope.

### EXAMPLE 7

### GA Activates a Heat Shock Response in Mammalian Cells

In order to induce a heat shock response COS-1 cells expressing the fusion of enhanced green fluorescent protein (EGFP) and the huntingtin exon 1 protein with 72 glutamines (H72Q) were treated with various concentrations of GA. Forty hours post transfection, total cell extracts were prepared and expression of EGFP-HD72Q and the heat shock proteins Hsp40, Hsp70 and Hsp90 was examined by immunoblot analysis using specific antibodies. As shown in FIG. 1A, soluble EGFP-HD72Q protein migrating in the SDS-gel at ˜57 kDa was detected in protein extracts prepared from transfected cells (lanes 1-6) but not in protein extracts of untransfected control cells (lane 7). Treatment of cells with increasing concentrations of GA (18-360 nM) had no effect on EGFP-HD72Q expression. In contrast, the expression of each of the molecular chaperones Hsp40, Hsp70 and Hsp90 increased with increasing GA-concentrations (lanes 1-4), indicating that treatment of cells with GA triggers a heat shock response. Addition of GA to a final concentration of 360 nM resulted in a 3-4-fold up-regulation of Hsp40, Hsp70 and Hsp90 compared to the untreated controls.

### EXAMPLE 8

### Activation of a Heat Shock Response by GA Inhibits Huntingtin Protein Aggregation

To determine whether induction of Hsp40, Hsp70, and Hsp90 expression by GA treatment has an effect on EGFP-HD72Q aggregation, COS-1 cells grown in the presence of various concentrations of GA were lysed and analyzed by a filter retardation assay for the presence of aggregated huntingtin protein (27). Using this assay SDS-resistant huntingtin protein aggregates can be immunologically detected and quantified. As shown in FIG. 1B, treatment of cells with GA resulted in a concentration-dependent inhibition of SDS-insoluble EGFP-HD72Q protein aggregates. At 18, 90, 180 and 360 nM, GA reduced the amount of insoluble protein aggregates by approximately 30, 60, 70 and 80%, respectively, as detected by the filtration assay.

The results obtained by the filter retardation assay were confirmed by fluorescence microscopy. Whereas in untreated control cells (FIG. 2A and B) large perinuclear EGFP-HD72Q protein aggregates with a diameter of 2-5 μm were detected, these structures were no longer visible in GA treated cells (FIG. 2C-F). At GA concentrations of 18-90 nM the large perinuclear inclusion bodies were replaced by smaller dot-like protein aggregates (diameter, 0.1-0.5 μm) that were dispersed throughout the cytoplasm. At higher GA concentrations (180-360 nM) these smaller aggregates were no longer detectable indicating that GA is a potent inhibitor of huntingtin protein aggregation in mammalian cells.

### EXAMPLE 9

### Hsp40 and Hsp70 Co-localize with Mutant Huntingtin in GA Treated Cells

To examine whether the molecular chaperones Hsp40, Hsp70 and Hsp90 co-localize with mutant huntingtin protein, GA treated COS-1 cells were permeabilized and analyzed by indirect immunofluorescence microscopy. Comparison of the fluorescence of EGFP-HD72Q with the immunostaining of Hsp40 and Hsp70 revealed that both chaperones co-localize with the mutant huntingtin protein (FIG. 3 A-F); At a GA concentration of 360 nM, EGFP-HD72Q as well as the chaperones Hsp40 and Hsp70 were evenly distributed in the cytoplasm and no perinuclear inclusion bodies with aggregated huntingtin protein were observed. Interestingly, under the same conditions, fluorescence of EGFP-HD72Q did only partially overlap with the immunostaining of Hsp90 (FIG. 3G-I), suggesting that a physical interaction of Hsp90 with the aggregation-prone huntingtin protein is not required to prevent aggregate formation. A direct interaction of Hsp40 and Hsp70 with EGFP-HD72Q, however appears to be critical for inhibition of polyglutamine assembly, consistent with previous findings (26).

### EXAMPLE 10

### Overexpression of Hsp70 and Hsp40 Inhibits HD Exon 1 Protein Aggregation in COS-1 Cells

To determine whether overexpression of heat shock proteins mimics the GA effect on huntingtin protein aggregation, the Flag- and HA-tagged heat shock proteins Hdj1 (Hsp40) and Hsp70, respectively, were transiently co-expressed with mutant HD51Q protein in COS-1 cells. Protein extracts were prepared 40 h post transfection and analyzed by SDS-PAGE and immunoblotting. As shown in FIG. 4A, the recombinant proteins HDQ51, Flag-Hdj1 and HA-Hsp70 migrating in the SDS-gel at about 30, 40 and 73 kDa, respectively, were detected in transfected but not in untransfected cells. In transfected cells both HA-Hsp70 and Flag-Hdj1 chaperones were overexpressed approximately 4-fold compared to the endogenous levels (data not shown). The effect of chaperone overexpression on HD51Q aggregation is shown in FIG. 4B. Co-expression of either Flag-Hdj1 or HA-Hsp70 with HD51Q resulted in an approximately 30-40% reduction of the amount of SDS-insoluble huntingtin aggregates in COS-1 cells. In comparison, when both chaperones were simultaneously co-expressed with HD51Q the amount of insoluble aggregates formed was diminished by 60-80%, indicating that a cooperation between Flag-Hdj1 and HA-Hsp70 is required for an efficient inhibition of HD51Q aggregation in COS-1 cells. Co-expression of Hsp90 with HD51Q had no discernible effect on the formation of insoluble protein aggregates suggesting that this chaperone is not directly involved in the inhibition of huntingtin protein aggregation in mammalian cells (data not shown).

Analysis by indirect immunofluorescence microscopy revealed that neither the overexpression of Flag-Hjd1 nor that of HA-Hsp70 was able to prevent the accumulation of large perinuclear inclusions with aggregated HD51Q protein (FIG. 5A-F). In strong contrast, when both chaperones were co-expressed with HD51Q the large perinuclear inclusion bodies totally disappeared and smaller dot-like aggregates with a diameter of 0.2-0.5 μm were observed (FIG. 5G-I). These aggregates were dispersed throughout the cytoplasm and were structurally similar to the ones observed after treatment of COS-1 cells with lower concentrations (18-90 nM) of geldanamycin (FIGS. 2C and D).

### EXAMPLE 11

### Overexpression of Hsp70 and Hsp40 Prevents Formation of Fibrillar Protein Aggregates

As morphological changes of protein aggregates in cells are poorly detectable by immunofluorescence microscopy, we also examined the effect of chaperone overexpression on aggregate formation by electron microscopy. At the ultrastructural level, most cells expressing HD51Q contained large perinuclear inclusion bodies (diameter 1-5 μm) composed of electron-dense filamentous material (FIG. 6A-C). The identity of the HD51Q fibrils was confirmed by immunoelectron microscopy using the anti-huntingtin antibodies AG51 (FIG. 6A and B) or HD1 (not shown) and a gold colloid secondary antibody. Interestingly, the anti-AG51 antibody immunolabeled mainly the periphery but not the interior of the inclusion bodies, suggesting that the HD exon 1 protein in the inclusion bodies is so densely packed that it is no longer accessible for the antibodies. Both Flag-Hdj1 and HA-Hsp70 co-localized with the perinuclear inclusion bodies; however, their association did not significantly alter the fibrillar structure of the HD51Q protein aggregates (FIG. 6D-E). As expected, in cells co-expressing Flag-Hdj1 and HA-Hsp70 no perinuclear HD51Q inclusion bodies were detected, once again indicating that overexpression of both heat shock proteins suppresses aggregate formation . Although more than 500 different cells co-expressing Flag-Hdj1/HA-Hsp70/HD51Q were examined by immunoelectron microscopy, in none of these cells large inclusion bodies with aggregated HD51Q protein could be observed. The mutant HD51Q protein appeared to be distributed homogenously in the cytoplasm of the transfected cells (FIG. 6F).

### References

1. HDCRG. (1993) A novel gene containing a trinucleotide repeat that is expanded and unstable on Huntington's disease chromosomes. The Huntington's Disease Collaborative Research Group. Cell, 72(6), 971-983.

2. DiFiglia, M., Sapp, E., Chase, K. O., Davies, S. W., Bates, G. P., Vonsattel, J. P. and Aronin, N. (1997) Aggregation of huntingtin in neuronal intranuclear inclusions and dystrophic neurites in brain. Science, 277(5334), 1990-1993.

3. Sieradzan, K. A., Mechan, A. O., Jones, L., Wanker, E. E., Nukina, N. and Mann, D. M. (1999) Huntington's disease intranuclear inclusions contain truncated, ubiquitinated huntingtin protein. Exp Neurol, 156(1), 92-99.

4. Davies, S. W., Turmaine, M., Cozens, B. A., DiFiglia, M., Sharp, A. H., Ross, C. A., Scherzinger, E., Wanker, E. E., Mangiarini, L. and Bates, G. P. (1997) Formation of neuronal intranuclear inclusions underlies the neurological dysfunction in mice transgenic for the HD mutation. Cell, 90(3), 537-548.

5. Wanker, E. E. (2000) Protein aggregation in Huntington's and Parkinson's disease: implications for therapy. Mol Med Today, 6(10), 387-391.

6. Saudou, F., Finkbeiner, S., Devys, D. and Greenberg, M. E. (1998) Huntingtin acts in the nucleus to induce apoptosis but death does not correlate with the formation of intranuclear inclusions. Cell, 95(1), 55-66.

7. Klement, I. A., Skinner, P. J., Kaytor, M. D., Yi, H., Hersch, S. M., Clark, H. B., Zoghbi, H. Y. and Orr, H. T. (1998) Ataxin-1 nuclear localization and aggregation: role in polyglutamine-induced disease in SCA1 transgenic mice. Cell, 95(1), 41-53.

8. Scherzinger, E., Sittler, A., Schweiger, K., Heiser, V., Lurz, R., Hasenbank, R., Bates, G. P., Lehrach, H. and Wanker, E. E. (1999) Self-assembly of polyglutamine-containing huntingtin fragments into amyloid-like fibrils: implications for Huntington's disease pathology. Proc Natl Acad Sci USA, 96(8), 4604-4609.

9. Hollenbach, B., Scherzinger, E., Schweiger, K., Lurz, R., Lehrach, H. and Wanker, E. E. (1999) Aggregation of truncated GST-HD exon 1 fusion proteins containing normal range and expanded glutamine repeats. Philos Trans R Soc Lond B Biol Sci, 354(1386), 991-994.

10. Heiser, V., Scherzinger, E., Boeddrich, A., Nordhoff, E., Lurz, R., Schugardt, N., Lehrach, H. and Wanker, E. E. (2000) Inhibition of huntingtin fibrillogenesis by specific antibodies and small molecules: implications for Huntington's disease therapy. Proc Natl Acad Sci USA, 97(12), 6739-6744.

11. Cummings, C. J., Mancini, M. A., Antalffy, B., DeFranco, D. B., Orr, H. T. and Zoghbi, H. Y. (1998) Chaperone suppression of aggregation and altered subcellular proteasome localization imply protein misfolding in SCA1. Nat Genet, 19(2), 148-154.

12. Satyal, S. H., Schmidt, E., Kitagawa, K., Sondheimer, N., Lindquist, S., Kramer, J. M. and Morimoto, R. I. (2000) Polyglutamine aggregates alter protein folding homeostasis in Caenorhabditis elegans. Proc Natl Acad Sci USA, 97(11), 5750-5755.

13. Krobitsch, S. and Lindquist, S. (2000) Aggregation of huntingtin in yeast varies with the length of the polyglutamine expansion and the expression of chaperone proteins. Proc Natl Acad Sci USA, 97(4), 1589-1594.

14. Chan, H. Y., Warrick, J. M., Gray-Board, G. L., Paulson, H. L. and Bonini, N. M. (2000) Mechanisms of chaperone suppression of polyglutamine disease: selectivity, synergy and modulation of protein solubility in drosophila. Hum Mol Genet, 9(19), 2811-2820.

15. Jana, N. R., Tanaka, M., Wang, G. and Nukina, N. (2000) Polyglutamine length-dependent interaction of Hsp40 and Hsp70 family chaperones with truncated N-terminal huntingtin: their role in suppression of aggregation and cellular toxicity [In Process Citation]. Hum Mol Genet, 9(13), 2009-2018.

16. Supko, J. G., Hickman, R. L., Grever, M. R. and Maispeis, L. (1995) Preclinical pharmacologic evaluation of geldanamycin as an antitumor agent. Cancer Chemother Pharmacol, 36(4), 305-315.

17. Chavany, C., Mimnaugh, E., Miller, P., Bitton, R., Nguyen, P., Trepel, J., Whitesell, L., Schnur, R., Moyer, J. and Neckers, L. (1996) p185erbB2 binds to GRP94 in vivo. Dissociation of the p185erbB2/GRP94 heterocomplex by benzoquinone ansamycins precedes depletion of p185erbB2. J Biol Chem, 271(9), 4974-4977.

18. Smith, D. F., Whitesell, L., Nair, S. C., Chen, S., Prapapanich, V. and Rimerman, R. A. (1995) Progesterone receptor structure and function altered by geldanamycin, an Hsp90-binding agent. Mol Cell Biol, 15(12), 6804-6812.

19. Xu, Y. and Lindquist, S. (1993) Heat-shock protein Hsp90 governs the activity of pp60v-src kinase. Proc Natl Acad Sci USA, 90(15), 7074-7078.

20. Whitesell, L., Mimnaugh, E. G., De Costa, B., Myers, C. E. and Neckers, L. M. (1994) Inhibition of heat shock protein HSP90-pp60v-src heteroprotein complex formation by benzoquinone ansamycins: essential role for stress proteins in oncogenic transformation. Pro Natl Acad Sci USA, 91(18), 8324-8328.

21. Prodromou, C., Roe, S. M., O'Brien, R., Ladbury, J. E., Piper, P. W. and Pearl, L. H. (1997) Identification and structural characterization of the ATP/ADP-binding site in the Hsp90 molecular chaperone. Cell, 90(1), 65-75.

22. Stebbins, C. E., Russo, A. A., Schneider, C., Rosen, N., Hartl, F. U. and Pavletich, N. P. (1997) Crystal structure of an Hsp90-geldanamycin complex: targeting of a protein chaperone by an antitumor agent. Cell, 89(2), 239-250.

23. Nathan, D. F. and Lindquist, S. (1995) Mutational analysis of Hsp90 function: interactions with a steroid receptor and a protein kinase. Mol Cell Biol, 15(7), 3917-3925.

24. Schneider, C., Sepp-Lorenzino, L., Nimmesgern, E., Ouerfelli, O., Danishefsky, S., Rosen, N. and Harti, F. U. (1996) Pharmacologic shifting of a balance between protein refolding and degradation mediated by Hsp90. Proc Natl Acad Sci USA, 93(25), 14536-14541.

25. Zou, J., Guo, Y., Guettouche, T., Smith, D. F. and Voellmy, R. (1998) Repression of heat shock transcription factor HSF1 activation by HSP90 (HSP90 complex) that forms a stress-sensitive complex with HSF1. Cell, 94(4), 471-480.

26. Muchowski, P. J., Schaffar, G., Sittler, A., Wanker, E. E., Hayer-Hartl, M. K. and Hartl, F. U. (2000) Hsp70 and Hsp40 chaperones can inhibit self-assembly of polyglutamine proteins into amyloid-like fibrils. Proc Natl Acad Sci USA, 97(14), 7841-7846.

27. Wanker, E. E., Scherzinger, E., Heiser, V., Siftler, A., Eickhoff, H. and Lehrach, H. (1999) Membrane filter assay for detection of amyloid-like polyglutamine-containing protein aggregates. Methods Enzymol, 309, 375-386.

28. Morimoto, R. I., Kroeger, P. E. and Cotto, J. J. (1996) The transcriptional regulation of heat shock genes: a plethora of heat shock factors and regulatory conditions. Exs, 77, 139-163.

29. DiFiglia, M. (1997) Clinical Genetics, II. Huntington's disease: from the gene to pathophysiology. Am J Psychiatry, 154(8), 1046.

30. Scherzinger, E., Lurz, R., Turmaine, M., Mangiarini, L., Hollenbach, B., Hasenbank, R., Bates, G. P., Davies, S. W., Lehrach, H. and Wanker, E. E. (1997) Huntingtin-encoded polyglutamine expansions form amyloid-like protein aggregates in vitro and in vivo. Cell, 90(3), 549-558.

31. Kastner, P., Perez, A., Lutz, Y., Rochette-Egly, C., Gaub, M. P., Durand, B., Lanotte, M., Berger, R. and Chambon, P. (1992) Structure, localization and transcriptional properties of two classes of retinoic acid receptor alpha fusion proteins In acute promyelocytic leukemia (APL): structural similarities with a new family of oncoproteins. Embo J, 11(2), 629-642.

32. Kozak, M. (1987) An analysis of 5′-noncoding sequences from 699 vertebrate messenger RNAs. Nucleic Acids Res, 15(20), 8125-8148.

33. Minami, Y., Hohfeld, J., Ohtsuka, K. and HartI, F. U. (1996) Regulation of the heat-shock protein 70 reaction cycle by the mammalian DnaJ homolog, Hsp40. J Biol Chem, 271(32), 19617-19624.

34. Gorman, C. M., Lane, D. P. and Rigby, P. W. (1984) High efficiency gene transfer into mammalian cells. Philos Trans R Soc Lond B Biol Sci, 307(1132), 343-346.

35. Sittler, A., Walter, S., Wedemeyer, N., Hasenbank, R., Scherzinger, E., Eickhoff, H., Bates, G. P., Lehrach, H. and Wanker, E. E. (1998) SH3GL3 associates with the Huntingtin exon 1 protein and promotes the formation of polygin-containing protein aggregates. Mol Cell, 2(4), 427-436.

36. Walter, S., Boeddrich, A., Lurz, R., Scherzinger, E., Gerhild, L., Lehrach, H. and Wanker, E. E. (2001) Accumulation of mutant huntingtin fragments in aggresome, like inclusion bodies as a result of insufficient protein degradation. Mol Biol Cell, in press.

37. Cho, C. Y., Moran, E. J., Cherry, S. R., Stephans, J. C., Fodor, S. P., Adams, C. L., Sundaram, A., Jacobs, J. W., and Schultz, P. G. (1993). An unnatural biopolymer, Science, 261, 1303-1305.

38. Fodor, S. P., Read, J. L., Pirrung, M. C., Stryer, L., Lu, A. T., and Solas, D. (1991). Light-directed, spatially addressable parallel chemical synthesis, Science 251, 767-773.

39. Joyce, G. F., and Inoue, T. (1989). A novel technique for the rapid preparation of mutant RNAs, Nucleic Acids Res. 17, 711-722.

40. Taylor, J. W., Ott, J., and Eckstein, F. (1985). The rapid generation of oligonucleotides-directed mutations at high frequency using phosphorothioate-modified DNA, Nucleic Acids Res. 13, 8765-8785.

41. Vandeyar, M. A., Weiner, M. P., Hutton, C. J., and Batt, C. A. (1988). A simple and rapid method for the selection of oligodeoxynucleotide-directed mutants, Gene 65, 129-133.

1. A method of treating a disease associated with protein aggregation and amyloid formation, comprising:
administering to a subject to prevent or treat the disease a compound or a plurality of compounds that
(a) inhibit function of Hsp90;
(b) inhibit binding of HSF1 to Hsp90; or
(c) activate expression of both Hsp40 and Hsp70 wherein said disease is Creutzfeld Jakob disease, spinal muscular atrophy, dentarorubral pallidoluysian atrophy, spinocerebellar ataxia type-1, -2, -3, -6 or -7, BSE, primary systemic amyloidosis, secondary systemic amyloidosis, senile systemic amyloidosis, familial amyloid polyneuropathy I, hereditary cerebral amyloid angiopathy, hemodialysis-related amyloidosis, familial amyloid polyneuropathy III, Finnish hereditary systemic amyloidosis, type II diabetes, medullary carcinoma of the thyroid, spongiform encephalopathies: Kuru, Gerstmann-Strälussler-Scheinker syndrome (GSS), familial insomnia, scrapie, atrial amyloidosis, hereditary non-neuropathic systemic amyloidosis, injection-localized amyloidosis or hereditary renal amyloidosis, and wherein said compound is selected from Herbimycin A, Novobiocin, 17-Allylamino, 17-demethoxygeldanamycin, macbecin, geldanamycin, radicicol and derivatives thereof.

2. The method of claim 1 wherein said disease is associated with polyglutamine expansions.

3. The method of claim 1 wherein said compound is geldanamycin.

4. The method of claim 1 wherein said plurality of compounds comprises geldanamycin.

5. The method of claim 1 wherein said compound or one of said compounds comprised in said plurality of compounds is derived from geldanamycin by
(a) modeling geldanamycin by peptidomimetics; and
(b) chemically synthesizing the modelled compound.

6. The method of claim 1 wherein said compound or one of said compounds comprised in said plurality of compounds are derived from geldanamycin by modification to achieve at least one property selected from the group consisting of:
modified site of action, spectrum of activity, organ specificity;
improved potency;
decreased toxicity (improved therapeutic index);
decreased side effects;
modified onset of therapeutic action, duration of effects;
modified pharmakinetic parameters (resorption, distribution, metabolism and excretion);
modified physico-chemical parameters (solubility, hygroscopicity, color, taste, odor, stability, state);
improved general specificity, organ/tissue specificity; and
optimized application form and route
by a method selected from the group consisting of:
esterification of carboxyl groups;
esterification of hydroxyl groups with carbon acids;
esterification of hydroxyl groups to, e.g. phosphates, pyrophosphates or sulfates or hemi succinates;
formation of pharmaceutically acceptable salts;
formation of pharmaceutically acceptable complexes;
synthesis of pharmacologically active polymers;
introduction of hydrophilic moieties;
introduction/exchange of substituents on aromates or side chains, change of substituent pattern;
modification by introduction of isosteric or bioisosteric moieties;
synthesis of homologous compounds;
introduction of branched side chains;
conversion of alkyl substituents to cyclic analogues;
derivatisation of hydroxyl group to ketales, acetales;
N-acetylation to amides, phenylcarbamates;
synthesis of Mannich bases, imines; and
transformation of ketones or aldehydes to Schiff's bases, oximes, acetales, ketales, enolesters, oxazolidines, thiozolidines;
or combinations thereof.

7. The method of claim 1 wherein said compound is obtained by
(a) screening an at least partially randomized peptide library and/or chemical compound library for molecules that
(aa) inhibit function of Hsp90; or
(ab) inhibit binding of HSF 1 to Hsp90; or
(ac) activate the expression of both Hsp40 and Hsp70, and optionally
(b) repeating step (a) one or more times.

8. The method of claim 7 wherein inhibition or activation of said Hsp,90, Hsp,40 or Hsp70 is assayed by a method selected from the group consisting of Reporter assays, immunofluorescence microscopy, a filter retardation assay and ATPase assays.

9. The method of claim 7 wherein the following further steps are conducted for obtaining said compound:
(c) modeling said compound by peptidomimetics; and
(d) chemically synthesizing the modeled compound.

10. The method of claim 7 wherein said compound is further modified to achieve at least one property selected from the group consisting of:
modified site of action, spectrum of activity, organ specificity;
improved potency;
decreased toxicity (improved therapeutic index);
decreased side effects;
modified onset of therapeutic action, duration of effect;
modified pharmakinetic parameters (resorption, distribution, metabolism and excretion);
modified physico-chemical parameters (solubility, hygroscopicity, color, taste, odor, stability, state);
improved general specificity, organ/tissue specificity; and
optimized application form and route
by a method selected from the group consisting of:
esterification of carboxyl groups;
esterification of hydroxyl groups with carbon acids;
esterification of hydroxyl groups, (e.g. to phosphates, pyrophosphates or sulfates or hemi succinates);
formation of pharmaceutically acceptable salts;
formation of pharmaceutically acceptable complexes;
synthesis of pharmacologically active polymers;
introduction of hydrophilic moieties;
introduction/exchange of substituents on aromates or side chains, change of substituent pattern;
modification by introduction of isosteric or bioisosteric moieties;
synthesis of homologous compounds;
introduction of branched side chains;
conversion of alkyl substituents to cyclic analogues;
derivatisation of hydroxyl group to ketales, acetales;
N-acetylation to amides, phenylcarbamates;
synthesis of Mannich bases, imines; and
transformation of ketones or aldehydes to Schiff's bases, oximes, acetales, ketales, enolesters, oxazolidines, thiozolidines or combinations thereof.

11. A method of designing a drug for the treatment of a disease associated with protein aggregation and amyloid formation wherein said disease is Creutzfeld Jakob disease, spinal muscular atrophy, dentarorubral pallidoluysian atrophy, spinocerebellar ataxia type-1, -2, -3, -6 or -7, BSE, primary systemic amyloidosis, secondary systemic amyloidosis, senile systemic amyloidosis, familial amyloid polyneuropathy I, hereditary cerebral amyloid angiopathy, hemodialysis-related amyloidosis, familial amyloid polyneuropathy III, Finnish hereditary systemic amyloidosis, type II diabetes, medullary carcinoma of the thyroid, spongiform encephalopathies: Kuru, Gerstmann-Sträussler-Scheinker syndrome (GSS), familial insomnia, scrapie, atrial amyloidosis, hereditary non-neuropathic systemic amyloidosis, injection-localized amyloidosis or hereditary renal amyloidosis comprising
(aa) identifying a site(s) of a compound that bind(s) to heat shock proteins 40 and/or 70; or
(ab) identifying a site(s) of a compound that bind(s) to the heat shock protein Hsp90 or to HSF1 and/or homologues thereof or other components participating in the regulation of the stress protein response;
(b) molecular modeling of both the binding site(s) in the compound and the heat shock protein(s); and
(c) modifying the compound to improve its binding specificity for the heat shock protein(s) or HSF1.

12. The method of claim 11 wherein identification of binding site(s) in step (a) is performed by site-directed mutagenesis or chimeric protein studies or a combination thereof.

13. The method of claim 11 wherein the compound is the compound as described in claim 1.

14. A method of identifying an activator of the expression of heat shock proteins 40 and/or 70 comprising
(a) testing a compound for the activation of translation wherein said compound is selected from geldanamycin, radicicol and derivatives thereof; or
(b) testing a compound for the activation of transcription wherein said compound binds to the promoter region of the genes encoding said heat shock protein(s) and preferably with transcription factors and responsive elements thereof; and
(c) selecting a compound that tests positive in (a) or (b).

15. A method of identifying an inhibitor of Hsp90 function comprising
(a) testing a compound for inhibition of Hsp90 ATPase activity function wherein said compound is selected from small molecules or peptides; and
(b) selecting a compound that tests positive in (a).

16. A method of identifying an inhibitor of binding of HSF1 to Hsp90 comprising
(a) testing a compound for inhibition of binding of HSF1 to Hsp90; and
(b) selecting a compound that tests positive in (a).

17. The method of any one of claims 14 to 16 further comprising
(a) modeling said compound by peptidomimetics; and
(b) chemically synthesizing the modeled compound.

18. The method of any one of claims 13 to 16 wherein said compound is further modified to achieve at least one property selected from the group consisting of:
modified site of action, spectrum of activity, organ specificity;
improved potency;
decreased toxicity (improved therapeutic index);
decreased side effects;
modified onset of therapeutic action, duration of effect;
modified pharmakinetic, parameters (resorption, distribution, metabolism and excretion);
modified physico-chemical parameters (solubility, hygroscopicity, color, taste, odor, stability, state);
improved general specificity, organ/tissue specificity; and
optimized application form and route by a method selected from the group consisting of:
esterification of carboxyl groups;
esterification of hydroxyl groups with carbon acids;
esterification of hydroxyl groups to, e.g. phosphates, pyrophosphates or sulfates or hemi succinates;
formation of pharmaceutically acceptable salts;
formation of pharmaceutically acceptable complexes;
synthesis of pharmacologically active polymers;
introduction of hydrophilic moieties;
introduction/exchange of substituents on aromates or side chains, change of substituent pattern;
modification by introduction of isosteric or bioisosteric moieties;
synthesis of homologous compounds;
introduction of branched side chains;
conversion of alkyl substituents to cyclic analogues;
derivatisation of hydroxyl group to ketales, acetales;
N-acetylation to amides, phenylcarbamates;
synthesis of Mannich bases, imines; and
transformation of ketones or aldehydes to Schiff's bases, oximes, acetates, ketales, enolesters, oxazolidines, thiozolidines or combinations thereof.

19. A method of producing a pharmaceutical composition comprising formulating the compound described in the method of any one of claims 1, 11, 14, 15, or 16 with a pharmaceutically acceptable carrier or diluent.

20. The method of any one of claims 1, 11, 14, 15, or 16, wherein said heat shock protein is/said heat shock proteins are human heat shock protein(s).

21. The method of claim 20 wherein the human heat shock protein 40 is Hdj-1 or Hdj-2.
